(19) United States
(12) Patent Application Publication
(10) Pub. No.: US 2006/0067981 A1
(43) Pub. Date: Mar. 30, 2006

(54) Contact lens with improved biocidal activity and related methods and materials

(75) Inventor: Erning Xia, Penfield, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/144,316

(22) Filed: Jun. 3, 2005

Related U.S. Application Data
(60) Provisional application No. 60/614,369, filed on Sep. 29, 2004.

(51) Int. Cl. A61F 2/00

(52) U.S. Cl. 424/428

(57) ABSTRACT

The present invention is directed to a contact lens comprising a porous lens material and a disinfectant, including but not limited to domiphen bromide. The disinfectant is typically absorbed into the pores of the lens. The present invention also includes a related process for manufacture, method of use and kit.

### CROSS REFERENCE

This application claims the benefit of Provisional Patent Application No. 60/614,369 filed Sep. 29, 2004 and is incorporated herein by reference.

### BACKGROUND OF THE INVENTION

1. Field of the Invention

This invention relates to a contact lenses and more particularly to lenses with biocidal properties.

2. Discussion of the Related Art

Contact lenses require disinfection before being inserted into the eye to prevent infecting the eye with microbes. Immersing, washing or soaking the contact lens in a cleaning solution that contains an antimicrobial typically accomplishes this.

Biguanide antimicrobials such as alexidine and PHMB are widely used as antimicrobials in ophthalmic solutions. For example, contact lens cleaning, disinfecting and have been commercialized in various products, typically at levels of about 1 ppm or less for use with soft contact lenses. Biguinides are polymerized so that the size of the biguanide molecules reduces the likelihood that the biguanide molecules will be absorbed into the pores of the lens. U.S. Pat. Nos. 5,358,688 and 5,536,861 teach antimicrobial quaternary ammonium group containing polymers that prevent infection and do not easily absorb into the pores of the lens material.

The use of antimicrobials can reduce the chance of a lens contaminated with microbes to be inserted into the eye. However, washing the lens with a disinfectant containing solution will not prevent infections with antimicrobials much long after the contact lens has been inserted into the eye of a patient.

Preventing an infection in the eye after the contact lens has been introduced into the eye of a patient is desirable. While any eye can be infected with a microbe, a contact lens may at least in some instances aggravate an eye infection. Regardless, it is desirable to have a contact lens that can reduce the chance of infection and/or disinfect the eye after it has been inserted into the eye of a patient.

U.S. Publ. No. 2003/0117579 teaches a medical device including a contact lens material that has an antimicrobial coating that comprises at least one layer of polymeric quaternary ammonium group containing compounds. The antimicrobial coating will only have a chance of killing microbes that contact the surface of the lens. Thus, the antimicrobial activity does not extend beyond the physical dimensions of the contact lens.

U.S. Publication No. 2003/0113291 teaches biocidal polymers that are the product of polycondensation of guanidine acid addition salt with diamines, which include polyalkylchains between the two amino groups. The antimicrobial material is throughout the polymer. Likewise, because the guanidine substituents are bound to the polymer, the antimicrobial activity does not extend beyond the limitations of the material.

U.S. Pat. No. 5,683,709 teaches a polymer resin comprising benzalkonium units in an amount to inhibit growth of microbes in the aqueous drug solution that is free of soluble quaternary ammonium salts. Similarly, the antimicrobial activity in situ does not extend beyond the polymer resin.

While significant improvements have been made in the disinfection of lenses in-situ, there still exists a need for an improved lens that disinfects in-situ and whose disinfecting abilities extend beyond the actual dimensions of the contact lens. The present invention addresses these and other needs.

### SUMMARY OF THE INVENTION

The present invention comprises a contact lens comprising a porous lens material and a disinfectant. Typically the disinfectant is contained within the pores of the porous lens material. When placed in the eye, the disinfectant is released into the fluid of the eye disinfecting the ocular region beyond the physical constraints of the lens. Generally, the disinfectant is selected to have biocidal properties but low toxicity on the tissue of the eye. Preferably, the disinfectant does not cause irritation in the eye of a patient.

The weight of the disinfectant in the porous lens material in one embodiment is a minimum of about 0.0001 μg of disinfectant and a maximum of about 100 μg of disinfectant for every mg of dry porous lens material. The term, “dry” as it relates to a lens material means a lens material that has no water. Thus, the expression “based upon [a specified weight] of dried lens material means that the weight of any lens wet lens material is adjusted to reduce the weight by the amount of free and bound water associated with the lens.

In one embodiment, there is a contact lens comprising a porous lens material and a disinfectant within pores in the porous lens material. Each mg of contact lens releases a disinfectant at a minimum rate of about 0.01 ng/day, about 0.1 ng/day, about 1 ng/day, about 10 ng/day and/or a maximum rate of about 100 ng/day, about 10 ng/day, about 1 ng/day.

In one embodiment, there is a contact lens that comprises a contact lens made from a porous lens material and a domiphen salt contained within the lens. In another embodiment, there is a kit comprising a vessel that contains an aqueous solution of a domiphen salt and a contact lens made from a porous lens material.

In another embodiment, there is a process for manufacturing a contact lens. The process comprises forming the contact lens from a porous lens material and placing the lens in a solution of a domiphen salt.

In still another embodiment, there is a process for killing microbes on the surface of the eye comprising the step of releasing a disinfectant into the eye from pores of a contact lens made of a porous material in a therapeutically effective amount for a minimum of about 1 week, about 2 weeks, about 4 weeks, about 8 weeks, or about 12 weeks.

In one embodiment, there is a contact lens comprising a porous lens material and a therapeutically active agent, wherein the disinfectant is released in a therapeutically effective amount over a maximum period of 12 weeks.

In another embodiment, there is a process for killing microbes on the surface of the eye comprising the step of releasing a therapeutic agent into the eye from the pores of a contact lens in a therapeutically effective amount over a minimum period of one week.

Other advantages and features will be apparent from the below detailed description of the invention, examples and drawings.

### BRIEF DESCRIPTION OF THE DRAWING

FIG. 1 is the uptake curve of Formula 1 in lens material.

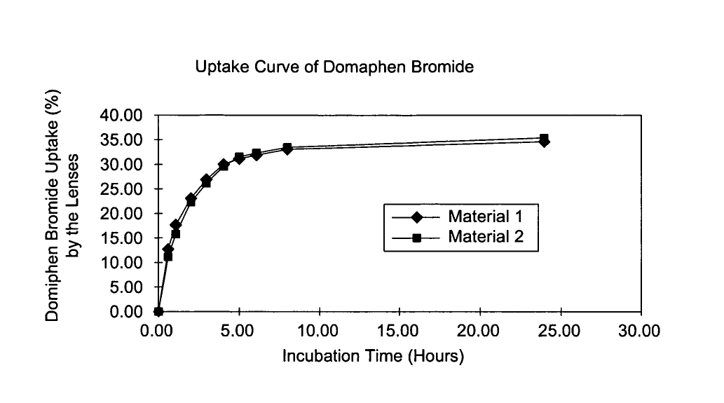

### DETAILED DESCRIPTION OF THE INVENTION

The present invention comprises a contact lens comprising a porous lens material and a disinfectant. Typically the disinfectant is contained within the pores of the porous lens material. When placed in the eye, the disinfectant is released into the fluid of the eye disinfecting the ocular region beyond the physical constraints of the lens. Generally, the disinfectant is selected to have biocidal properties but low toxicity on the tissue of the eye. Preferably, the disinfectant does not cause irritation in the eye of a patient. Formula of the Present Invention

The present invention comprises in one embodiment a contact lens that is loaded with a disinfectant within the pores of the lens material. In one embodiment of disinfectant in the porous lens material is a minimum of about 0.0001 μg of disinfectant and a maximum of about 100 μg of disinfectant for every mg of dry porous lens material. In an embodiment, the weight of disinfectant in the porous lens material is a minimum of about 0.005 μg, about 0.001 μg or about 0.0005 μg for every mg of dry porous lens material. Optionally, the weight of disinfectant in the porous lens material is a maximum of about 50 μg, about 10 μg, 5 μg, 1 μg or about 0.5 μg for every mg of dry porous lens material.

Lens Material

The porous lens material is a hydrogel material. In one embodiment, the hydrogel is a silicone hydrogel. In another embodiment, the hydrogel is a fluorosilicone hydrogel. In still another embodiment the hydrogel is made of a material selected from the group consisting of siloxy-containing monomers, siloxy-containing monomers, alkyl-, cycloalkyl-, arylalkyl- and aryl-containing derivatives or isoprenes. Such systems could also be copolymerized with hydrophilic monomers such as 2-hydroxyethyl methacrylate, N-vinylpyrrolidene, N,N-dimethylacrylamide and (meth)acrylic acid. In still another embodiment, the contact lens is a rigid gas permeable lens.

In another embodiment, the contact lens material is a hydrogel material. Typically, the porous lens material is a silicone hydrogel material—preferably a fluorosilicone hydrogel material. In one embodiment, the porous lens material is selected from the group consisting of siloxy-containing monomers, siloxy-containing monomers, alkyl-, cycloalkyl-, arylalkyl- and aryl-containing derivatives or isoprenes. Such systems could also be copolymerized with hydrophilic monomers such as 2-hydroxyethyl methacrylate, N-vinylpyrrolidene, N,N-dimethylacrylamide and (meth)acrylic acid.

Hydrogels comprise hydrated, cross-linked polymeric systems containing water in an equilibrium state. Conventional hydrogel lens materials include polymers containing monomers such as 2-hydroxyethyl methacrylate (HEMA), glyceryl methacrylate, N-vinylpyrrolidone (NVP) and dimethacrylamide.

Flexible ophthalmic lens materials useful in the present invention include silicone hydrogels as well as conventional hydrogels and low-water elastomeric materials. Examples of flexible ophthalmic lens materials useful in the present invention are taught in U.S. Pat. No. 5,908,906 to Künzler et al.; U.S. Pat. No. 5,714,557 to Künzler et al.; U.S. Pat. No. 5,710,302 to Künzler et al.; U.S. Pat. No. 5,708,094 to Lai et al.; U.S. Pat. No. 5,616,757 to Bambury et al.; U.S. Pat. No. 5,610,252 to Bambury et al.; U.S. Pat. No. 5,512,205 to Lai; U.S. Pat. No. 5,449,729 to Lai; U.S. Pat. No. 5,387,662 to Künzler et al. and U.S. Pat. No. 5,310,779 to Lai; which patents are incorporated by reference as if set forth at length herein.

U.S. Pat. Nos. 6,037,328, 6,008,317, 5,981,675, 5,981,669, 5,969,076, 5,945,465, 5,914,355, 5,858,937, 5,824,719 and 5,726,733 teach ophthalmic lens materials containing HEMA monomers.

U.S. Pat. Nos. 6,071,439, 5,824,719, 5,726,733, 5,708,094, 5,610,204, 5,298,533, 5,270,418, 5,236,969 and 5,006,622 teach ophthalmic lens materials containing glyceryl methacrylate monomers.

U.S. Pat. Nos. 6,008,317, 5,969,076, 5,908,906, 5,824,719, 5,726,733, 5,714,557, 5,710,302, 5,708,094, 5,648,515 and 5,639,908 teach ophthalmic lens materials containing NVP monomers.

U.S. Pat. Nos. 5,539,016, 5,512,205, 5,449,729, 5,387,662, 5,321,108 and 5,310,779 teach ophthalmic lens materials containing dimethacrylamide monomers.

The preferred conventional hydrogel materials typically contain HEMA, NVP and TBE (4-t-butyl-2-hydroxycyclohexyl methacrylate). Polymacon™ materials, for example the Soflens 66™ brand contact lenses (commercially available from Bausch & Lomb Incorporated of Rochester, N.Y.) are examples of particularly preferred conventional hydrogel materials.

Silicone hydrogels generally have a water content greater than about five weight percent and more commonly between about ten to about eighty weight percent. Materials are usually prepared by polymerizing a mixture containing at least one silicone-containing monomer and at least one hydrophilic monomer. Either the silicone-containing monomer or the hydrophilic monomer may function as a cross-linking agent (a cross-linker being defined as a monomer having multiple polymerizable functionalities) or a separate cross-linker may be employed. Applicable silicone-containing monomeric units for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779 and 5,358,995.

A preferred silicone hydrogel material comprises (in the bulk monomer mixture that is copolymerized) 5 to 50 percent, preferably 10 to 25, by weight of one or more silicone macromonomers, 5 to 75 percent, preferably 30 to 60 percent, by weight of one or more polysiloxanylalkyl(meth)acrylic monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729 and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

Suitable hydrophilic monomers include those monomers that, once polymerized, can form a complex with poly(acrylic acid). The suitable monomers form hydrogels useful in the present invention and include, for example, monomers that form complexes with poly(acrylic acid) and its derivatives. Examples of useful monomers include amides such as N,N-dimethyl acrylamide, N,N-dimethyl methacrylamide, cyclic lactams such as N-vinyl-2-pyrrolidone and poly(alkene glycol)s functionalized with polymerizable groups. Examples of useful functionalized poly(alkene glycol)s include poly(diethylene glycol)s of varying chain length containing monomethacrylate or dimethacrylate end caps. In a preferred embodiment, the poly(alkene glycol) polymer contains at least two alkene glycol monomeric units. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. In a particularly preferred embodiment, the hydrophilic monomers used in the contact lens material are capable of forming a stable complex with a cationic polysaccharide.

In one embodiment, the contact lens material is a rigid gas permealble lens made of a rigid gas permeable lens material. Rigid ophthalmic lens materials include rigid-gas-permeable (“RGP”) materials. RGP materials typically comprise a hydrophobic crosslinked polymer system containing less than 5 wt. % water. RGP materials useful in accordance with the present invention include those materials taught in U.S. Pat. No. 4,826,936 to Ellis; U.S. Pat. No. 4,463,149 to Ellis; U.S. Pat. No. 4,604,479 to Ellis; U.S. Pat. No. 4,686,267 to Ellis et al.; U.S. Pat. No. 4,826,936 to Ellis; U.S. Pat. No. 4,996,275 to Ellis et al.; U.S. Pat. No. 5,032,658 to Baron et al.; U.S. Pat. No. 5,070,215 to Bambury et al.; U.S. Pat. No. 5,177,165 to Valint et al.; U.S. Pat. No. 5,177,168 to Baron et al.; U.S. Pat. No. 5,219,965 to Valint et al.; U.S. Pat. No. 5,336,797 to McGee and Valint; U.S. Pat. No. 5,358,995 to Lai et al.; U.S. Pat. No. 5,364,918 to Valint et al.; U.S. Pat. No. 5,610,252 to Bambury et al.; U.S. Pat. No. 5,708,094 to Lai et al; and U.S. Pat. No. 5,981,669 to Valint et al. U.S. Pat. No. 5,346,976 to Ellis et al. teaches a preferred method of making an RGP material. The patents mentioned above are incorporated by reference as if set forth at length herein.

Disinfectant Composition

In one embodiment, the disinfectant is a quaternary ammonium salt.

Representative examples of the quaternary ammonium compounds are compositions comprised of benzalkonium halides or, for example, balanced mixtures of n-alkyl dimethyl benzyl ammonium chlorides. Other examples include polymeric quaternary ammonium salts used in ophthalmic applications such as poly[(dimethyliminio)-2-butene-1,4-diyl chloride], [4-tris(2-hydroxyethyl)ammonio]-2-butenyl-w-[tris(2-hydroxyethyl)ammonio]dichloride (chemical registry number 75345-27-6) generally available as Polyquaternium 1® from ONYX Corporation.

Representative biguanides are the bis(biguanides), such as alexidine or chlorhexidine or salts thereof, and polymeric biguanides such as polymeric hexamethylene biguanides (PHMB).

Polymeric hexamethylene biguanides (commercially available from Zeneca, Wilmington, Del.), their polymers and water-soluble salts being most preferred. Generally, the hexamethylene biguanide polymers, also referred to as polyaminopropyl biguanide (PAPB), have molecular weights of up to about 100,000. Such compounds are known and are disclosed in U.S. Pat. No. 4,758,595 which patent is incorporated herein be reference.

In one preferred embodiment, the disinfectant is selected from the group comprising polymeric hexamethylene biguanides (PHMB), alexidine 2HCl, a domiphen salt or CAE. In another embodiment, the disinfectant is a domiphen salt.

Typically, the concentration of the disinfectant, including but not limited to a domiphen salt in the porous lens material is a minimum of about 0.0001 μg of disinfectant and a maximum of about 100 μg of disinfectant for every mg of dry porous lens material.

Generally, wherein the disinfectant is loaded into the pores in a solution having a disinfectant concentration that is a minimum of about 0.0001 wt. %, about 0.001 wt. %, about 0.01 wt. % or about 0.1 wt. %. Generally, the disinfectant is loaded into the pores in a solution having a disinfectant concentration that is a maximum of about 0.01 wt. %, about 1 wt. % or about 10 wt. %.

The aqueous solutions of the present invention are typically adjusted with tonicity agents to approximate the tonicity of normal lacrimal fluids (approximately equivalent to a 0.9% solution of sodium chloride or 3% glycerol solution). The solutions are made substantially isotonic with physiological saline used alone or in combination with other adjusting agents. The ophthalmic compositions preferably have an osmolality of about 225 mOsm/kg to 400 mOsm/kg, more preferably 280 mOsm/kg to 320 mOsm/kg.

The compositions may include chelating or sequestering agents in order to chelate or bind metal ions, which might otherwise react with the lens and/or protein deposits and collect on the lens. Examples of such preferred materials, may include, but are not limited to ethylene-diaminetetraacetic acid (EDTA) and its salts (disodium), which are usually added in amounts ranging from about 0.01 weight percent to about 0.2 weight percent.

Compositions, such as aqueous solutions, for use in the present invention, may be formulated as lens conditioning solutions or eye-drops and sold in a wide range of small-volume containers from 1 ml to 30 ml in size. Such containers can be made from HDPE (high density polyethylene), LDPE (low density polyethylene), polypropylene, poly(ethylene terepthalate) and the like. For eye drops, flexible bottles having conventional dispensing tops are especially suitable for use with the present invention. The eye-drop formulation of the invention is used by instilling, for example, about one (1) or three (3) drops in the eye(s) as needed.

The pH of the solutions and/or compositions of the present invention may be maintained within the range of pH=5.0 to 8.0, preferably about pH=6.0 to 8.0, more preferably about pH=6.5 to 7.8, most preferably pH values of greater than or equal to 7; suitable buffers may be added, such as borate, citrate, bicarbonate, tris(hydroxymethyl)aminomethane (TRIS) and various mixed phosphate buffers (which may include combinations of Na₂HPO₄, NaH₂PO₄ and KH₂PO₄) and mixtures thereof. Borate buffers are preferred when the primary antimicrobial agent is PAPB. Generally, buffers will be used in amounts ranging from about 0.05 percent by weight to 2.5 percent by weight, and preferably, from 0.1 percent by weight to 1.5 percent weight.

Surfactants, which are suitable for use in the present invention, are classified into cationic surfactants, anionic surfactants, nonionic surfactants and ampholytic surfactants depending upon their dissociation state in their aqueous solutions. Among them, various surfactants, which are classified into cationic surfactants, particularly surfactants which consist of an amino acid derivative, i.e. amino acid type cationic surfactants, have conventionally been proposed as disinfectant cleaning agents or compositions for disinfection. Glycerin may also be included as a component of the present invention. Amphoteric surfactants suitable for use in a composition according to the present invention include materials of the type are offered commercially under the trade name “Miranol.” Another useful class of amphoteric surfactants is exemplified by cocoamidopropyl betaine, commercially available from various sources. Various other surfactants suitable for use in the composition can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Optionally, one or more additional polymeric or non-polymeric demulcents may be combined with the above-named ingredients. Demulcents are known to provide wetting, moisturizing and/or lubricating effects, resulting in increased comfort. Polymeric demulcents can also act as a water-soluble viscosity builder. Included among the water-soluble viscosity builders are the non-ionic cellulosic polymers like methyl cellulose, hydroxyethyl cellulose, hydroxypropyl cellulose, and carboxymethyl cellulose, poly(N-vinylpyrrolidone), poly(vinylalcohol) and the like. Such viscosity builders or demulcents may be employed in a total amount ranging from about 0.01 to about 5.0 weight percent or less. Suitably, the viscosity of the final formulation is 10 cps to 50 cps. Comfort agents such as glycerin or propylene glycol can also be added.

Process for Making a Contact Lens

In one embodiment, there is a process for manufacturing a contact lens comprising the steps of forming the contact lens from a porous lens material. The contact lens is soaked in a solution comprising an aqueous medium and a disinfectant. The period of soaking is a minimum of about 4 hours, about 8 hours, about 12 hours, about 24 hours, about 2 days or about 1 week. The period of soaking is a maximum of the shelf life of the contact lens product. In one embodiment, the period of soaking is a maximum of about 1 year, about six months, about two months, about one month, about 2 weeks, about 1 week or about 4 days. For reusable lenses the period for soaking is a maximum of about 2 weeks, about 1 week, about 4 days, about 1 day, about 18 hours, about 12 hours, about 8 hours or about 4 hours.

In one embodiment, large amounts of disinfectant are released initially into the eye in the first 12 hours to 24 hours. The initial release of large amounts can be prevented by soaking the contact lenses into a regular solution, coating solution, and/or an artificial tears during manufacturing process.

The disinfectant compositions of this invention can be prepared by a variety of techniques conventionally used in the art. One method involves a two-phase compounding procedures. In the first phase, about 30 percent of the distilled water is used to dissolve the polymeric components (such as the cationic, cellulosic polymer) with mixing for about 30 minutes at around 50° C. The first-phase solution is then autoclaved at about 120° C. for 30 minutes. In a second phase, other components, such as alkali metal chlorides, sequestering agents, preservatives and buffering agents, are then dissolved in about 60 percent of the distilled water with agitation, followed by adding the balance of distilled water. The second-phase solution can then be sterilely added into the first-phase solution by forcing it through a 0.22 micron filter by means of pressure, followed by packaging in sterilized plastic containers.

The materials suitable for use in the present invention may also be useful as a component of a cleaning, disinfecting or conditioning solution and/or composition. Such solutions and/or compositions also may include, antimicrobial agents, surfactants, toxicity adjusting agents, buffers and the like that are known to be used components of conditioning and/or cleaning solutions for contact lenses. Examples of suitable formulations for cleaning and/or disinfecting solutions are taught in U.S. Pat. No. 5,858,937 to Richard et al., which is incorporated by reference as if set forth at length herein.

Methods of Use

In one embodiment, the process kills microbes on the surface of the eye comprising the step of releasing a disinfectant into the eye from pores of a contact lens made of a porous material in a therapeutically effective amount over a period of a minimum of about one week, two weeks, four weeks or eight weeks. In one embodiment, the process kills microbes on the surface of the eye comprising the step of releasing a disinfectant into the eye from pores of a contact lens made of a porous material in a therapeutically effective amount over a period of a minimum of about one week, two weeks, four weeks, eight weeks or twelve weeks.

In one embodiment, the lens is preferably rinsed before being inserted into the eye of a patient. The rinse is a hypotonic sterile aqueous solution. In another embodiment, the rinse is a hypotonic saline solution. In another embodiment, the rinse is distilled water.

In one embodiment, there is a contact lens comprising a porous lens material and a disinfectant within pores in the porous lens material. Each mg of contact lens releases a disinfectant at a minimum rate of about 0.01 ng/day, about 0.1 ng/day, about 1 ng/day, about 10 ng/day and/or a maximum rate of about 100 ng/day, about 10 ng/day, about 1 ng/day.

In one embodiment, there is a process for killing microbes on the surface of the eye comprising the step of releasing a therapeutic agent into the eye from the pores of a contact lens in a therapeutically effective amount over a minimum period of one week.

Product System or Kit

According to one embodiment, a kit comprising a vessel that contains an aqueous solution of a domiphen salt and a contact lens made from a porous lens material.

According to one embodiment, the lens includes any lens that is set forth in the present invention.

### EXAMPLES

### Example 1

### Test Formulations

One or more of the following composition was prepared to test the biocidal effect of domiphen bromide.

TABLE 1
Composition of Test Formulations

| Ingredient/Property | % W/W |
|---|---|
| Sodium Borate | 0.09 |
| Boric Acid | 0.85 |
| Sodium Chloride | 0.45 |
| Domiphen Bromide | 10 ppm-100 ppm |
| pH = 7.0 | |
| Osmo.(mOsmo/Kg) = 270 | |

### Example 2

### Biocidal Test Results

A test was conducted to study the microbiocidal efficacy of solutions prepared according to the present invention with TEA as compared to the same solutions prepared without TEA. Three test solutions were prepared in accordance with the Test Formulation that was identified above as Table 1 with domaphen bromide concentrations of 10 ppm (Solution 1), 30 ppm (Solution 2) and 90 ppm (Solution 3). The antimicrobial efficacy of Solutions 1-3 for the chemical disinfection of contact lenses was evaluated. Microbial challenge inoculums were prepared using Pseudomonas aeruginosa (ATCC 9027), Staphylococcus aureus (ATCC 6538), Serratia marcescens (ATCC 13880), Candida albicans (ATCC 10231) and Fusarium solani (ATCC 36031).

The test organisms were cultured on appropriate agar and the cultures were harvested using sterile Dulbecco's Phosphate Buffered Saline plus 0.05 percent weight/volume polysorbate 80 (DPBST) or a suitable diluent and transferred to a suitable vessel. Spore suspensions were filtered through sterile glass wool to remove hyphal fragments. Serratia marcescens, as appropriate, was filtered through a 1.2 micron filter to clarify the suspension. After harvesting, the suspension was centrifuged at no more than 5000× gravitational force for a maximum of 30 minutes at 20 to 25 degrees Celsius. The supernatent was poured off and suspended in DPBST or other suitable diluent. The suspension was centrifuged a second time, and resuspended in DPBST or other suitable diluent. All challenge bacterial and fungal cell suspensions were adjusted with DPBST or other suitable diluent to 1×10⁷ to 1×10⁸ cfu/mL.

The appropriate cell concentration is estimated by measuring the turbidity of the suspension. For example, a spectrophotometer is used at 490 nm. One tube was prepared containing a minimum of 10 mL of test solution per challenge organism. Each tube of the solution to be tested was inoculated with a suspension of the test organism sufficient to provide a final count of 1×10⁵ to 1×10⁶ cfu/mL. The volume of the inoculum did not exceed 1 percent of the sample volume. Dispersion of the inoculum was ensured by vortexing the sample for at least 15 seconds. The inoculated product was stored between 10 and 25 degrees Celsius.

Aliquots of the inoculated product were taken in the amount of 1.0 mL for determination of viable counts after certain time periods of disinfection. The time points for the bacteria were, for example, 1, 2, 3 and 4 hours when the proposed regimen soaking time was four hours. Yeast and mold were tested at an additional timepoint of 16 hours (4 times the regimen time). The suspension was mixed well by vortexing vigorously for at least 5 seconds. The 1.0 mL aliquots removed at the specified time intervals were subjected to a suitable series of decimal dilutions in validated neutralizing media. The suspensions were mixed vigorously and incubated for a suitable period of time to allow for neutralization of the microbial agent. The viable count of organisms was determined in appropriate dilutions by preparation of triplicate plates of trypticase soy agar (TSA) for bacteria and Sabouraud dextrose agar (SDA) for mold and yeast. The bacterial recovery plates were incubated at 30 to 35 degrees Celsius for two to four days.

The yeast recovery plates were incubated at 20 to 30 degrees Celsius for two to four days. The mold recovery plates were incubated at 20 to 25 degrees Celsius for three to seven days. The average number of colony forming units was determined on countable plates. Countable plates refer to 30 to 300 cfu/plates for bacteria and yeast, and 8 to 80 cfu/plates for mold except when colonies are observed only for the 10⁰ or 10⁻¹ dilution plates. The microbial reduction was then calculated at the specified time points and recorded as set forth below in Table 2.

In order to demonstrate the suitability of the medium used for growth of test organisms and to provide an estimation of the initial inoculum concentration, inoculum controls were made by dispersing an identical aliquot of the inoculum into a suitable diluent, for example DPBST, using the same volume of diluent used to suspend the organism listed above. Following inoculation in a validated neutralizing broth and incubation for an appropriate period of time, the inoculum control must be between 1.0×10⁵ to 1.0×10⁶ cfu/mL. Formula one was evaluated based on the performance requirement referred to as the “Stand-Alone Procedure for Disinfecting Products” (Stand-Alone Test) and is based on the Disinfection Efficacy Testing for contact lens care products under the Premarket Notification (510(k)) Guidance Document for Contact Lens Care Products dated May 1, 1997, prepared by the U.S. Food and Drug Administration, Division of Ophthalmic Devices.

This performance requirement does not contain a rub procedure. This performance requirement is comparable to current ISO standards for disinfection of contact lenses (revised 1995). The Stand-Alone Test challenges a disinfecting product with a standard inoculum of a representative range of microorganisms and establishes the extent of viability loss at predetermined time intervals comparable with those during which the product may be used. The primary criteria for a given disinfection period, corresponding to a potential minimum recommended disinfection period, is that the number of bacteria recovered per mL must be reduced by a mean value of not less than 3.0 logs within the given disinfection period. The number of mold and yeast recovered per ml must be reduced by a mean value of not less than 1.0 log within the minimum recommended disinfection time with no increase at four times the minimum recommended disinfection time.

TABLE 2
Biocidal Test Results

| | | Solution 1 | Solution 2 | Solution 3 |
|---|---|---|---|---|
| | Domiphen Bromide | (10 ppm) | (30 ppm) | (90 ppm) |
| S. aureus | 30 minutes | 3.4 | >4.8 | >4.8 |
| | 60 minutes | >4.8 | >4.8 | >4.8 |
| | 4 hours | >4.8 | >4.8 | >4.8 |
| P. aeruginosa | 30 minutes | 4.2 | >4.7 | >4.7 |
| | 60 minutes | >4.7 | >4.7 | >4.7 |
| | 4 hours | 4.5 | >4.7 | >4.7 |
| S. marcescens | 30 minutes | 3.5 | >4.8 | >4.8 |
| | 60 minutes | >4.8 | >4.8 | >4.8 |
| | 4 hours | >4.8 | >4.8 | >4.8 |
| C. albicans | 30 minutes | 2.7 | 4.7 | 4.7 |
| | 60 minutes | 4.3 | >4.7 | >4.7 |
| | 4 hours | >4.7 | >4.7 | >4.7 |
| A. niger | 30 minutes | 3.1 | >4.4 | >4.4 |
| | 60 minutes | 4.1 | >4.4 | >4.4 |
| | 4 hours | >4.4 | >4.4 | >4.4 |

The above data regarding the biocidal efficacy of domiphen bromide shows that Solution 1 with a concentration as low as 10 ppm domaphen bromide was efficacious against S. marcesens (ATCC#13880), C. albican (ATCC#10231) and F. solani (ATCC#36031).

### Example 3

### Uptake of Domaphen Bromide in Tris(hydroxymethyl)aminomethane

Buffer System

Three solutions made according to the formulation of Example 1 were made with a 25 ppm (Solution 4), 50 ppm (Solution 5) and 100 ppm (Solution 6) domaphen bromide concentrations, except the borate buffer system was replaced with an equivalent amount of a tris(hydroxymethyl)aminomethane buffer system and the water amount was adjusted accordingly. The term “equivalent amount” means the amount required to adjust the pH to the desired pH as another buffer.

Two 3 ml portions of Solutions 4-6 in solution were placed in six separate vials. A total of six Sureview™ lens (Johnson & Johnson, New Brunswick, N.J.) were placed in the vials—one lens per each vial. The vials remained in the lens for a first 24 hour soaking cycle. After 24 hours, the lenses were removed. The amount of domaphen bromide remaining in each of the 3 ml solutions is measured by ultraviolet visible (UV/VIS) spectrometer analytical method. Inhibition % is calculated to reflect the amount of domaphen bromide that remains in the solution expressed as a percentage of the total amount of domaphen bromide. Inhibition % is recorded in Table 3 corresponding to a soaking time of 24 hours. This completed first test cycle.

The lenses removed during the first soaking cycle were placed in six fresh vials containing two 3 ml portions of solutions 4-6 to commence the second 24 hour soaking cycle. Care was taken to make sure that the solution the lens is soaked in during the second cycle is identical to the solution in the first cycle. Thus, each pair of lenses is soaked in identical concentrations of Solutions 4-6. After 24 hours, the lenses are removed. The solutions are tested to determine the concentration domaphen bromide in solution. The inhibition % is calculated and recorded in Table 3 corresponding to 48 hour soak time.

The lenses were again soaked for a third and fourth soak cycle. The domaphen bromide inhibition % was recorded after the third and fourth soak-cycle and recorded in Table 3 corresponding to the 72 hour and 96 hour soak times, respectively.

TABLE 3
Domiphen Bromide Conc. (ppm) in Tris Buffer

| | Inhibition % in 25 | Inhibition % in 50 | Inhibition % in 100 |
|---|---|---|---|
| Soaking | ppm Domiphen | ppm Domiphen | ppm Domiphen |
| Time | Bromide | Bromide | Bromide |
| 24 | 78.59 | 73.59 | 68.39 |
| | 75.76 | 67.65 | 63.58 |
| 48 | 62.72 | 66.35 | 63.71 |
| | 57.91 | 68.18 | 65.50 |
| 72 | 59.19 | 58.25 | 56.54 |
| | 53.50 | 56.17 | 58.19 |
| 96 | 55.13 | 80.84 | 53.91 |
| | 52.65 | 81.31 | 59.25 |

### Example 4

### Uptake of Domaphen Bromide in Phosphate Buffer System

Three solutions made according to the formulation of Example 1 were made with a 25 ppm (Solution 7), 50 ppm (Solution 8) and 100 ppm (Solution 9) domaphen bromide concentrations, except the borate buffer system was replaced with an equivalent amount of a phosphate buffer system and the amount of water was adjusted accordingly.

Two 3 ml portions of Solutions 7-9 in solution were placed in six separate vials. A total of six Sureview™ lens (Johnson & Johnson, New Brunswick, N.J.) were placed in the vials—one lens per each vial. The vials remained in the lens for a first 24 hour soaking cycle. After 24 hours, the lenses were removed. The amount of domaphen bromide remaining in each of the 3 ml solutions is measured by ultraviolet visible (UV/VIS) spectrometer analytical method. Inhibition % is calculated to reflect the amount of domaphen bromide that remains in the solution expressed as a percentage of the total amount of domaphen bromide. Inhibition % is recorded in Table 4 corresponding to a soaking time of 24 hours. This completed the first test cycle.

The lenses removed during the first soaking cycle were placed in six fresh vials containing two 3 ml portions of solutions 7-9 to commence the second 24 hour soaking cycle. Care was taken to make sure that the solution the lens is soaked in during the second cycle is identical to the solution in the first cycle. Thus, each pair of lenses is soaked in concentrations of Solutions 7-9 identical to the concentration that the lens was soaked in during the first soak cycle. After 24 hours, the lenses are removed. The solutions are tested to determine the concentration domaphen bromide in solution. The inhibition % is calculated and recorded in Table 4 corresponding to 48 hour soak time.

The lenses were again soaked for a third and fourth soak cycle. The domaphen bromide inhibition % was recorded after the third and fourth soak-cycle and recorded in Table 4 corresponding to the 72 hour and 96 hour soak times, respectively.

TABLE 4
Domiphen Bromide Conc. (ppm) in Phosphate Buffer

| | Inhibition % in 25 | Inhibition % in 50 | Inhibition % in 100 |
|---|---|---|---|
| Soaking | ppm Domiphen | ppm Domiphen | ppm Domiphen |
| Time | Bromide | Bromide | Bromide |
| 24 | 43.92 | 47.12 | 48.87 |
| | 51.40 | 49.78 | 47.95 |
| 48 | 51.75 | 49.86 | 47.52 |
| | 50.19 | 49.51 | 46.77 |
| 72 | 52.34 | 46.67 | 43.55 |
| | 47.54 | 48.35 | 45.54 |
| 96 | 41.31 | 34.47 | 34.67 |
| | 34.37 | 37.30 | 37.15 |

### Example 5

### Uptake of Domaphen Bromide in Citrate Buffer System

Three solutions made according to the formulation of Example 1 were made with a 25 ppm (Solution 10), 50 ppm (Solution 11) and 100 ppm (Solution 12) domaphen bromide concentrations, except the borate buffer system was replaced with an equivalent amount of a citrate buffer system and the water amount was adjusted accordingly.

Two 3 ml portions of Solutions 10-12 in solution were placed in six separate vials. A total of six Sureview™ lens (Johnson & Johnson, New Brunswick, N.J.) were placed in the vials—one lens per each vial. The vials remained in the lens for a first 24 hour soaking cycle. After 24 hours, the lenses were removed. The amount of domaphen bromide remaining in each of the 3 ml solutions is measured by ultraviolet visible (UV/VIS) spectrometer analytical method. Inhibition % is calculated to reflect the amount of domaphen bromide that remains in the solution expressed as a percentage of the total amount of domaphen bromide. Inhibition % is recorded in Table 5 corresponding to a soaking time of 24 hours. This completed the first test cycle.

The lenses removed during the first soaking cycle were placed in six fresh vials containing two 3 ml portions of solutions 10-12 to commence the second 24 hour soaking cycle. Care was taken to make sure that the solution the lens is soaked in during the second cycle is identical to the solution in the first cycle. Thus, each pair of lenses is soaked in concentrations of Solutions 10-12 identical to the concentration that the lens was soaked in during the first soak cycle. After 24 hours, the lenses are removed. The solutions are tested to determine the concentration domaphen bromide in solution. The inhibition % is calculated and recorded in Table 5 corresponding to 48 hour soak time.

The lenses were again soaked for a third and fourth soak cycle. The domaphen bromide inhibition % was recorded after the third and fourth soak cycle and recorded in Table 5 corresponding to the 72 hour and 96 hour soak times, respectively.

TABLE 5
Domiphen Bromide Conc. (ppm) in Citrate Buffer

| | Inhibition % in 25 | Inhibition % in 50 | Inhibition % in 100 |
|---|---|---|---|
| Soaking | ppm Domiphen | ppm Domiphen | ppm Domiphen |
| Time | Bromide | Bromide | Bromide |
| 24 | 63.20 | 76.51 | 75.59 |
| | 75.66 | 80.02 | 77.95 |
| 48 | 73.25 | 72.39 | 70.90 |
| | 71.57 | 78.39 | 79.63 |
| 72 | 64.34 | 67.91 | 71.04 |
| | 76.59 | 77.42 | 81.39 |
| 96 | 59.17 | 59.28 | 62.97 |
| | 64.89 | 69.87 | 74.47 |

### Example 6

### Uptake of Domaphen Bromide in Borate Buffer System

Three solutions made according to the formulation of Example 1 were made with a 25 ppm (Solution 13), 50 ppm (Solution 14) and 100 ppm (Solution 15) domaphen bromide concentrations.

Two 3 ml portions of Solutions 13-15 in solution were placed in six separate vials. A total of six Sureview™ lens (Johnson & Johnson, New Brunswick, N.J.) were placed in the vials—one lens per each vial. The vials remained in the lens for a first 24 hour soaking cycle. After 24 hours, the lenses were removed. The amount of domaphen bromide remaining in each of the 3 ml solutions is measured by ultraviolet visible (UV/VIS) spectrometer analytical method. Inhibition % is calculated to reflect the amount of domaphen bromide that remains in the solution expressed as a percentage of the total amount of domaphen bromide. Inhibition % is recorded in Table 6 corresponding to a soaking time of 24 hours. This completed the first test cycle.

The lenses removed during the first soaking cycle were placed in six fresh vials containing two 3 ml portions of Solutions 13-15 to commence the second 24 hour soaking cycle. Care was taken to make sure that the solution the lens is soaked in during the second cycle is identical to the solution in the first cycle. Thus, each pair of lenses is soaked in concentrations of Solutions 13-15 identical to the concentration that the lens was soaked in during the first soak cycle. After 24 hours, the lenses are removed. The solutions are tested to determine the concentration domaphen bromide in solution. The inhibition % is calculated and recorded in Table 6 corresponding to 48 hour soak time.

The lenses were again soaked for a third and fourth soak cycle. The domaphen bromide inhibition % was recorded after the third and fourth soak cycle and recorded in Table 6 corresponding to the 72 hour and 96 hour soak times, respectively.

TABLE 6
Domiphen Bromide Conc. (ppm) In Borate Buffer

| | Inhibition % in 25 | Inhibition % in 50 | Inhibition % in 100 |
|---|---|---|---|
| Soaking | ppm Domiphen | ppm Domiphen | ppm Domiphen |
| Time | Bromide | Bromide | Bromide |
| 24 | 21.85 | 29.80 | 14.68 |
| | 21.85 | 29.88 | 17.15 |
| 48 | 35.57 | 32.62 | 15.10 |
| | 36.68 | 36.31 | 19.08 |
| 72 | 33.41 | 31.35 | 12.02 |
| | 33.18 | 37.25 | 18.58 |
| 96 | 31.54 | 28.30 | 14.11 |
| | 39.03 | 37.79 | 20.21 |

Analysis of Examples 3-6 illustrate that the borate buffer was most effective with the domaphen bromide. The loading percentage, which is calculated as 100% minus Inhibition%, is as high as 70-80%.

The loading time for most of buffer system is about 12-24 hours by soaking commercial contact lenses inside the 3 ml contact cases with a certain concentration of domaphen bromide solution.

Although several specific embodiments have been depicted and described in detail, it will be apparent to those skilled in the relevant art that the specification including the examples are made without the intention of limiting the scope of the invention and that various modifications, additions, substitutions, and the like can be made without departing from the spirit of the invention are therefore considered to be within the scope of the invention as defined in the claims which follow.

What is claimed is:

1. A contact lens comprising a porous lens material and a disinfectant, wherein weight of disinfectant in the porous lens material is a minimum of about 0.0001 μg of disinfectant and a maximum of about 100 μg of disinfectant for every mg of dry porous lens material.

2. The contact lens of claim 1, wherein the porous lens material is a rigid gas permeable lens.

3. The contact lens of claim 1, wherein the porous lens material is a hydrogel material.

4. The contact lens of claim 1, wherein the disinfectant is a quaternary ammonium salt.

5. The contact lens of claim 1, wherein the disinfectant is a domiphen salt.

6. The contact lens of claim 6, wherein the concentration of domiphen bromide in the porous lens material is a minimum of about 0.0001 μg of domiphen bromide and a maximum of about 10 μg of domiphen bromide for every mg of dry porous lens material.

7. The contact lens of claim 1, wherein the disinfectant is loaded into the pores in a solution having a disinfectant concentration that is a minimum of about 0.0001 μg of disinfectant and a maximum of about 100 μg of disinfectant for every mg of dry porous lens material.

8. The contact lens of claim 1, wherein the porous lens material is selected from the group consisting of siloxy-containing monomers, siloxy-containing monomers, alkyl-, cycloalkyl-, arylalkyl- and aryl-containing derivatives or isoprenes. Such systems could also be copolymerized with hydrophilic monomers such as 2-hydroxyethyl methacrylate, N-vinylpyrrolidene, N,N-dimethylacrylamide and (meth)acrylic acid.

9. A contact lens comprising a porous lens material and a disinfectant within pores in the porous lens material, wherein the contact lens releases at a minimum rate of about 10 ng/day and a maximum rate of about 200 ng/day.

10. The contact lens of claim 9, wherein the porous lens material is a rigid gas permeable lens.

11. The contact lens of claim 9, wherein the porous lens material is a hydrogel material.

12. The contact lens of claim 9, wherein the disinfectant is a quaternary ammonium salt.

13. The contact lens of claim 9, wherein the disinfectant is a domiphen salt.

14. The contact lens of claim 13, wherein the concentration of domiphen salt in the porous lens material is a minimum of about 0.0001 μg of domiphen salt and a maximum of about 10 μg of domiphen salt for every mg of dry porous lens material.

15. The contact lens of claim 9, wherein the disinfectant is loaded into the pores in a solution having a disinfectant concentration that is a minimum of about 0.0001 μg of disinfectant and a maximum of about 100 μg of disinfectant for every mg of dry porous lens material.

16. The contact lens of claim 9, wherein the porous lens material of the contact lenses is selected from the group consisting of siloxy-containing monomers, siloxy-containing monomers, alkyl-, cycloalkyl-, arylalkyl- and aryl-containing derivatives or isoprenes.

17. A kit comprising a vessel that contains an aqueous solution of a domiphen salt and a contact lens made from a porous lens material.

18. The kit of claim 17, wherein the porous lens material is a rigid gas permeable lens.

19. The kit of claim 17, wherein the porous lens material is a hydrogel material.

20. The kit of claim 17, wherein the concentration of domiphen salt in the porous lens material is a minimum of about 0.0001 μg of domiphen salt and a maximum of about 10 μg of domiphen salt for every mg of dry porous lens material.

21. The kit of claim 17, wherein the disinfectant is loaded into the pores in a solution having a disinfectant concentration that is a minimum of about 0.0001 μg of disinfectant and a maximum of about 100 μg of disinfectant for every mg of dry porous lens material.

22. The kit of claim 17, wherein the porous lens material is selected from the group consisting of siloxy-containing monomers, siloxy-containing monomers, alkyl-, cycloalkyl-, arylalkyl- and aryl-containing derivatives or isoprenes.

23. The kit of claim 17, wherein the contact lens releases disinfectant from the pores at a minimum rate of about 10 ng/day and a maximum rate of about 2000 ng/day.

24. A process for manufacturing a contact lens comprising the steps of
forming the contact lens from a porous lens material;
placing the lens in a solution of a domiphen salt.

25. The process of claim 24, wherein the porous lens material is a rigid gas permeable lens.

26. The process of claim 24, wherein the porous lens material is a hydrogel material.

27. The process of claim 24, wherein the-concentration of domiphen salt in the porous lens material is a minimum of about 0.0001 μg of domiphen salt and a maximum of about 10 μg of domiphen salt for every mg of dry porous lens material.

28. The process of claim 24, wherein the disinfectant is loaded into the pores in a solution having a disinfectant concentration that is a is a minimum of about 0.0001 μg of disinfectant and a maximum of about 100 μg of disinfectant for every mg of dry porous lens material.

29. The process of claim 24, wherein the porous material of the contact lenses is selected from the group consisting of siloxy-containing monomers, siloxy-containing monomers, alkyl-, cycloalkyl-, arylalkyl- and aryl-containing derivatives or isoprenes.

30. The process of claim 24, wherein the contact lens releases disinfectant from the pores at a minimum rate of about 10 ng/day and a maximum rate of about 2000 ng/day.

31. A process for killing microbes on the surface of the eye comprising the step of releasing a disinfectant into the eye from pores of a contact lens made of a porous material in a therapeutically effective amount over a minimum period of one week.

32. The process of claim 31, wherein the porous lens material is a rigid gas permeable lens.

33. The process of claim 31, wherein the porous lens material is a hydrogel material.

34. The process of claim 31, wherein the disinfectant is a quaternary ammonium salt.

35. The process of claim 31, wherein the disinfectant is a domiphen salt.

36. The process of claim 35, wherein the concentration of domiphen salt in the porous lens material is a minimum of about 0.0001 μg of domiphen salt and a maximum of about 10 μg of domiphen salt for every mg of dry porous lens material.

37. The process of claim 31, wherein the disinfectant is loaded into the pores in a solution having a disinfectant concentration that is a minimum of about 0.0001 μg of disinfectant and a maximum of about 100 μg of disinfectant for every mg of dry porous lens material.

38. The process of claim 31, wherein the porous material of the contact lenses is selected from the group consisting of siloxy-containing monomers, siloxy-containing monomers, alkyl-, cycloalkyl-, arylalkyl- and aryl-containing derivatives or isoprenes.

39. The process of claim 31, wherein the contact lens releases disinfectant from the pores at a minimum rate of about 10 ng/day and a maximum rate of about 2000 ng/day.

40. A contact lens comprising a porous lens material and a therapeutically active agent, wherein the disinfectant is released in a therapeutically effective amount over a minimum period of one week.

41. The contact lens of claim 40, wherein the porous lens material is a rigid gas permeable lens.

42. The contact lens of claim 40, wherein the porous lens material is a hydrogel material.

43. The contact lens of claim 40, wherein the disinfectant is a quaternary ammonium salt.

44. The contact lens of claim 40, wherein the disinfectant is a domiphen salt.

45. The contact lens of claim 44, wherein the concentration of domiphen salt in the porous lens material is a minimum of about 0.0001 μg of domiphen salt and a maximum of about 10 μg of domiphen salt for every mg of dry porous lens material.

46. The contact lens of claim 40, wherein the disinfectant is loaded into the pores in a solution having a disinfectant concentration that is a minimum of about 0.0001 μg of disinfectant and a maximum of about 100 μg of disinfectant for every mg of dry porous lens material.

47. The contact lens of claim 40, wherein the porous material of the contact lenses is selected from the group consisting of siloxy-containing monomers, siloxy-containing monomers, alkyl-, cycloalkyl-, arylalkyl- and aryl-containing derivatives or isoprenes.

48. The contact lens of claim 40, wherein the contact lens releases disinfectant from the pores at a minimum rate of about 10 ng/day and a maximum rate of about 2000 ng/day.

49. A process for killing microbes on the surface of the eye comprising the step of releasing a therapeutic agent into the eye from the pores of a contact lens in a therapeutically effective amount over a minimum of one week.

50. The process of claim 49, wherein the porous lens material is a rigid gas permeable lens.

51. The process of claim 49, wherein the porous lens material is a hydrogel material.

52. The process of claim 49, wherein the disinfectant is a quaternary ammonium salt.

53. The process of claim 49, wherein the disinfectant is a domiphen salt.

54. The process of claim 49, wherein the concentration of domiphen salt in the porous lens material is a minimum of about 0.0001 μg of domiphen salt and a maximum of about 10 μg of domiphen salt for every mg of dry porous lens material.

55. The process of claim 49, wherein the disinfectant is loaded into the pores in a solution having a disinfectant concentration that is a minimum of about 0.0001 μg of disinfectant and a maximum of about 100 μg of disinfectant for every mg of dry porous lens material.

56. The process of claim 49, wherein the porous material of the contact lenses is selected from the group consisting of siloxy-containing monomers, siloxy-containing monomers, alkyl-, cycloalkyl-, arylalkyl- and aryl-containing derivatives or isoprenes.

57. The process of claim 49, wherein the contact lens releases disinfectant from the pores at a minimum rate of about 10 ng/day and a maximum rate of about 2000 ng/day.
